(19) United States
(12) Patent Application Publication
(10) Pub. No.: US 2023/0201297 A1
(43) Pub. Date: Jun. 29, 2023

(54) COMPOSITION COMPRISING SAFRANAL AND PROBIOTICS

(71) Applicant: ACTIV'INSIDE, BEYCHAC ET CAILLAU (FR)

(72) Inventors: David GAUDOUT, CARIGNAN DE BORDEAUX (FR); Stéphane REY, MONTELIMAR (FR); Benoit LEMAIRE, LIBOURNE (FR); Astrid DE VULPILLIERES, BORDEAUX (FR); Line POURTAU, CESTAS (FR)

(21) Appl. No.: 17/925,989

(22) PCT Filed: May 20, 2021
(86) PCT No.: PCT/EP2021/063519

§ 371 Date: Nov. 17, 2022

(30) Foreign Application Priority Data
May 20, 2020 (FR) FR2005201

(51) Int. Cl. A61K 36/896; A61K 35/744; A61K 9/50; A61K 9/00; A61P 1/00

(52) U.S. Cl. CPC A61K 36/896; A61K 35/744; A61K 9/50; A61K 9/0056; A61P 1/00

(57) ABSTRACT

The present invention relates to a composition comprising at least one plant extract containing at least 0.2% safranal, in particular derived from Crocus Sativus, and at least one probiotic bacterium and/or probiotic yeast, and uses thereof.

### SUMMARY OF THE INVENTION

The present invention relates to a composition comprising at least one plant extract comprising at least 0.2% safranal measured by HPLC, microencapsulated, in particular an extract of Crocus sativus, and at least one probiotic bacterium and/or probiotic yeast, as well as the uses thereof. The composition according to the invention can be used as a nutritional or pharmaceutical product in numerous applications, in particular in order to improve irritable bowel syndrome (IBS), sleep disorders, immune disorders, or stress in humans and animals.

### PRIOR ART

IBS is a chronic intestinal condition, the physiopathological mechanisms of which are not yet entirely known, and which affects 9 to 23% of the population (Saha L, “Irritable bowel syndrome: pathogenesis, diagnosis, treatment, and evidence-based medicine.” World J Gastroenterol. 2014 Jun. 14; 20(22):6759-73). It is a multifactorial condition combining gut hypersensitization, an abnormality of the gastric motility, and an alternation of the intestinal permeability.

It has recently been shown that disorders of the immune system, serotoninergic abnormalities in the region of the intestines, disturbances of the intestinal microbiota, as well as psychological factors such as stress or anxiety, can also promote the onset of IBS. The symptomatology of IBS manifests as abdominal pain or discomfort, bloating, as well as transit disorders such as diarrhea, constipation, or an alternation between the two. Together, the symptoms of IBS can significantly change the quality of life by causing sleep disorders (Fass R, Fullerton S, Tung S, Mayer E A. “Sleep disturbances in clinic patients with functional bowel disorders.” Am J Gastroenterol. 2000; 95: 1195-2000; Bellini M, Gemignani A, Gambaccini D, et al. “Evaluation of latent links between irritable bowel syndrome and sleep quality.” World J Gastroenterol. 2011; 17: 5089-5096) or a reduction in self-esteem.

Interestingly, it has been shown that sleep disorders were not only a consequence of IBS, but also a risk factor of the onset of the disorder (Creed F, “Review article: the incidence and risk factors for irritable bowel syndrome in population-based studies,” Aliment Pharmacol Ther. 2019 September; 50(5):507-516). Thus, the risk of developing IBS was 1.6 times greater in the case of people having sleep disorders, compared with individuals who do not have these (Vege S S, Locke G R 3rd, Weaver A L, Farmer S A, Melton L J 3rd, Talley N J. “Functional gastrointestinal disorders among people with sleep disturbances: a population-based study.” Mayo Clin Proc. 2004; 79:1501-1506).

A strong correlation was also observed between the severity of IBS and psychiatric disorders, in particular depression and anxiety. A review on the psychosocial determinants of IBS reports a significant increase in the stress score just before the onset of the disease (Surdea-Blaga T, B\check{a}ban A, Dumitrascu D L, “Psychosocial determinants of irritable bowel syndrome, World J Gastroenterol.” 2012 Feb. 21; 18(7):616-26).

These observations clearly show the existence of a communication system between the brain and the gut. Today, the underlying physiopathological mechanisms are known in part, but there is significant research remaining. Besides the humoral pathway, a gut-brain axis communication pathway is the neural pathway. Indeed, the digestive tract, in addition to its intrinsic innervation, communicates with the brain in a bidirectional manner, via the sympathetic and parasympathetic inputs and outputs.

The measurement of the activity of the automatic system (ANS), via the heart rate variability (HRV) (measurement frequently used to evaluate the overall activity of the ANS) has thus been subject to a plurality of studies in IBS. In particular, a reduction in the HRV in patients suffering from IBS has been demonstrated, compared with subjects in good health (Polster A, Friberg P, Gunterberg V, Öhman L, Le Nevé B, Tornblom H, Cvijovic M, Simren M, “Heart rate variability characteristics of patients with irritable bowel syndrome and associations with symptoms,” Neurogastroenterol Motil. 2018 July; 30(7)). Interestingly, a reduction in the HRV is also observed in subjects suffering depression, or in the case of stress (Hartmann R, Schmidt F M, Sander C, Hegerl U, “Heart Rate Variability as Indicator of Clinical State in Depression,” Front Psychiatry. 2019 Jan. 17; 9:735).

It is thus of great interest to develop solutions, acting on the communication pathways of the gut-brain axis, for preventing and combating the symptoms and the risk factors of IBS, in order to improve the quality of life of people suffering from this. Given that a disrupted intestinal microenvironment (intestinal dysbiosis), caused by chronic stress for example, promotes the development and persistence of IBS, today manipulation of the intestinal microbiota in order to improve the symptoms of IBS is part of new treatment strategies. A plurality of different approaches have been studied in order to improve the composition of the intestinal microbiota.

It has thus been shown that dietary changes, including supplementation with fiber, prebioitics and probiotics, improve the symptoms and the composition of the intestinal microbiota in IBS. In the field of research evaluating the effects of probiotics, the lactic acid bacteria, such as Lactobacillus and Bifidobacterium, have been studied most frequently.

It has also been shown that the use of a plurality of strains of the same species, or of a plurality of bacteria of different species, allowed for an improvement in overall symptoms of IBS compared with the use of strains in isolation (Ortiz-Lucas M, Tobias A, Saz P, Sebastien J J. “Effects of probiotic species on irritable bowel syndrome symptoms: a bring up to date metaanalysis.” Rev Esp Enferm Dig. 2013; 105(1): 19-36). The use of a probiotic containing Lactobacillus in turn allows a significant reduction in some specific symptoms such as abdominal pains or flatulence, and improves the quality of life (Asha M Z, Khalil S F H, Efficacy and Safety of Probiotics, Prebiotics and Synbiotics in the Treatment of Irritable Bowel Syndrome: A systematic review and meta-analysis, Sultan Qaboos Univ Med J. 2020 February; 20(1):e13-e24).

More particularly, it has been shown that the mixture of Bifidobacterium animalis, Lactobacillus acidophillus, Lactobacillus delbrueckii and Streptococcus thermophilus used at a dose of 4*10^{\hat{ }9} CFU, twice a day for a period of 4 weeks, made it possible to reduce the abdominal pain and bloating, and made possible a satisfactory alleviation of general symptoms compared with taking a placebo, in individual suffering from IBS (Jafari E, Vahedi H, Merat S et al. (2014) “Therapeutic effects, tolerability and safety of a multi-strain probiotic in Iranian adults with irritable bowel syndrome and bloating.” Arch Iran Med 17, 466-470).

Recent studies also demonstrated the interest in using probiotics in depression (Smith K S, Greene M W, Babu J R, Frugé AD, Psychobiotics as treatment for anxiety, depression, and related symptoms: a systematic review. Nutr Neurosci. 2019 Dec. 20:1-15), anxiety, stress or sleep. Other studies also evaluated the beneficial role of probiotics on the automatic nervous system, in particular on the HRV.

However, to date there is no recommendation in the prior art regarding specific probiotics to use for combating the symptoms of IBS (McKenzie Y A, Bowyer R K, Leach H, Gulia P, Horobin J, O'Sullivan N A, Pettitt C, Reeves L B, Seamark L, Williams M, Thompson J, Lomer M C; (IBS Dietetic Guideline Review Group on behalf of Gastroenterology Specialist Group of the British Dietetic Association, British Dietetic Association systematic review and evidence-based practice guidelines for the dietary management of irritable bowel syndrome in adults (2016 update), J Hum Nutr Diet. 2016 October; 29(5):549-75).

Some compounds or extracts of plant origin have also demonstrated an effectiveness on the symptoms or risk factors associated with IBS, in particular compounds or extracts of Crocus sativus.

The selective serotonin reuptake inhibitors (SSRIs) are anti-depressants used in the treatment of IBS. Of these, fluoxetine has demonstrated an effectiveness for a dose of 20 mg/day (Trinkley, K. E. & Nahata, M. C. “Medication management of irritable bowel syndrome.” Digestion 89, 253-267 (2014). However, the SSRIs are often associated with side-effects, whereas extracts of plant origin have a similar activity without causing undesirable effects. In a double-blind randomized clinical study, the addition of 30 mg per day of saffron extract demonstrated an effect equivalent to taking 20 mg fluoxetine, in terms of improvement of quality of life in 66 people suffering from IBS.

Saffron has also demonstrated its effectiveness on the risk factors associated with IBS, with the improvement of sleep quality (Shahdadi, H., Balouchi, A. & Dehghanmehr, S. Effect of saffron oral capsule on anxiety and quality of sleep of diabetic patients in a tertiary healthcare facility in southeastern Iran: A quasi-experimental study. Trop. J. Pharm. Res. 16, 2749-2753 (2017); Milajerdi, A. et al. The effects of alcoholic extract of saffron (Crocus sativus L.) on mild to moderate comorbid depression-anxiety, sleep quality, and life satisfaction in type 2 diabetes mellitus: A double-blind, randomized and placebo-controlled clinical trial. Complement Ther Med 41, 196-202 (2018)), the improvement of mood disorders (Akhondzadeh, S. et al. Crocus sativus L. in the treatment of mild to moderate depression: a double-blind, randomized and placebo-controlled trial. Phytother Res 19, 148-151 (2005).) as well as an increase in the activity of the immune system (Kianbakht, S. & Ghazavi, A. Immunomodulatory effects of saffron: A randomized double-blind placebo-controlled clinical trial. Phyther. Res. 25, 1801-1805 (2011)).

More precisely, the effects of saffron for minimizing the risk factors of developing IBS are associated with the action of safranal, as is reported by sleep (Liu, Z. et al. Safranal enhances non-rapid eye movement sleep in pentobarbital-treated mice. CNS Neurosci Ther 18, 623-630 (2012)), stress (Fukui, H., Toyoshima, K. & Komaki, R. Psychological and neuroendocrinological effects of odor of saffron (Crocus sativus). Phytomedicine 18, 726-730 (2011)) and immunity studies (Bukhari S I, Pattnaik B, Rayees S, Kaul S, Dhar M K, Safranal of Crocus sativus L. inhibits inducible nitric oxide synthase and attenuates asthma in a mouse model of asthma. Phytother Res. 29(4), 617-27 (2015)). In people subject to stress, adding 30 mg of saffron titered to 0.2% safranal demonstrated a beneficial role in maintaining the HRV.

However, there is still a significant need for a product which can prevent or treat IBS, sleep, immunity or stress disorders, with improved effectiveness compared with the prior art.

### SUMMARY OF THE INVENTION

The aim of the present invention is therefore that of meeting this need by proposing a novel composition comprising at least one specific microencapsulated plant extract comprising at least 0.2% safranal by weight with respect to the total weight of the dry matter, measured by HPLC method, and at least one probiotic bacterium and/or probiotic yeast, for preventing or treating mainly IBS, sleep, immunity and/or stress disorders.

In the prior art, effects on IBS, sleep, immunity and stress disorders of an extract of Crocus Sativus or of a probiotic bacterium, a yeast or a mixture of strains individually have indeed been suggested, but a composition of this kind, in particular when the extract is microencapsulated, has not been described.

However, surprisingly, although it is known in the prior art that saffron exhibits antimicrobial effects (Nanasombat et al. 2014; Liu et al. 2017; Nadir et al. 2019; Ambrosio et al. 2020), and therefore that saffron is a priori not compatible with the use of bacteria, the inventors observed that the specific combination of probiotic bacteria and/or yeasts and specific microencapsulated plant extract comprising safranal, such as saffron, made it possible to maintain the viability of the bacteria used in combination with the extract of Crocus sativus, and thus to achieve an improved effect on IBS, sleep, immunity and stress disorders in humans or animals. Moreover, the particular quantity of active molecules, and in particular of safranal, contained in the plant extract, also makes it possible to improve the effect of the composition.

Thus, the plant extract integrated into the composition according to the invention is an extract of Crocus sativus comprising at least 0.2% safranal by weight with respect to the total weight of the dry matter, measured by HPLC method.

The probiotic bacterium integrated into the composition according to the invention is preferably selected from the bacteria of the genus Lactobacillus, Bifidobacterium, Bacillus, Streptococcus, Enterococcus, Pediococcus and Escherichia.

The probiotic yeast integrated into the composition according to the invention is preferably selected from the yeasts of the genus Saccharomyces.

The invention is also aimed at non-therapeutic uses of the present composition Other features and advantages will become clear from the following detailed description of the invention, the examples, and the drawings.

### BRIEF DESCRIPTION OF THE FIGURES

FIG. 1 shows the kinetics of the growth of Lactobacillus delbrueckii in the presence of a microencapsulated saffron extract or crushed stigmata of Crocus Sativus containing at least 0.2% safranal.

FIG. 2 shows petri dishes of Lactobacillus delbrueckii (dilution 10^{\hat{ }6}), after 48 hours of incubation in the presence of two different plant extracts comprising safranal. A: in the presence of crushed stigmata of Crocus Sativus. B: in the presence of a microencapsulated saffron extract.

FIG. 3 shows the glucose/fructose consumption after 24 hours of incubation in the presence of a microencapsulated saffron extract or crushed stigmata of Crocus Sativus containing at least 0.2% safranal, ** p<0.01.

### DETAILED DESCRIPTION OF THE INVENTION

### I. Definitions

Within the meaning of the invention, “animal” means any animal except for human beings (humans).

Within the meaning of the invention, “nutritional composition” means any mixture of food compounds intended to be ingested and comprising at least 0.2% safranal measured by HPLC, in particular an extract of Crocus sativus, and at least one probiotic bacterium and/or probiotic yeast. The nutritional compositions are in particular food compositions or foods, or food supplements.

Within the meaning of the invention, “plant extract of a plant” means all or part of said plant or at least one molecule or a set of a plurality of molecules originating from all or some of a plant. This may be a specific selection of native molecules present in the plant, or molecules obtained by any kind of transformation of said native molecules. The raw material used for obtaining the extract may be formed of all or part of a plant containing safranal. If the plant containing safranal is Crocus sativus, the plant extract according to the invention may be all or part of the Crocus sativus or may be obtained in particular from stigmata and/or petals and/or bulbs of saffron, preferably from stigmata.

The extraction methods are well known to a person skilled in the art. In particular the method consisting in implementing the following steps can be cited: use of stigmata of Crocus sativus, followed by crushing using a pin mill, to a size of 250 μm, followed by hydroalcoholic extraction using ethanol 60% v/v at a rate of 50 g of saffron per liter of hydroalcoholic solution, followed by impregnation on maltodextrin, introduced into the hydroalcoholic solution, and heat treatment in a sterilizer for 48 hours at 40° C. Alternatively, the extraction may be an acidified aqueous extraction using hydrochloric acid of pH 4, followed by impregnation on gum arabic introduced into the aqueous solution, and heat treatment in a sterilizer for 72 hours at 40° C. Other methods are incorporated by reference and described in the patent FR 3054443.

Within the meaning of the invention, “impregnation on a support” means adding a bulking agent into the extraction solution. The support or bulking agent can in particular be selected from the following constituents: maltodextrin, sugar, silica, gum arabic, preferably maltodextrin. Following this impregnation step, the method may also comprise a step of emulsion and/or encapsulation of the obtained extract, preferably microencapsulation. This step consists in high-speed stirring of the extraction solution containing the bulking agent and optionally the auxiliary agent. It can in particular be performed using auxiliary agents such as gum arabic, cyclodextrins or fats, proteins, peptides.

Within the meaning of the invention, “microencapsulation on a support” means the insertion of active substances, in particular the extract of Crocus sativus according to the invention, with particles of sizes between 1 μm and 1000 μm, and more preferably between 30 μm and 500 μm, and even more preferably between 50 μm and 300 μm.

Within the meaning of the invention, “support” means any food substance of plant, mineral or chemical origin that is used as an ingredient or food additive and allows the impregnation, and thereby the dilution, of the extract of the invention. When the extract is impregnated on a support, the percentages of molecules contained in the extract are given by weight of dry matter of the extract including the support.

Within the meaning of the invention, “prevent” or “prevention” means the reduction of a given phenomenon to a lower level of risk or probability of occurrence, i.e., in the context of the present invention, for example IBS, a sleep, immunity, or stress disorder.

Within the meaning of the invention, “treat” or “treatment” means a reduction in the progression of the disease or the disorder, a stabilization, an inversion or regression, or indeed an interruption or inhibition of the progression of a disease or a disorder. In the context of the invention, these terms also apply to one or more symptoms of said diseases or disorders of the present invention.

### II. Composition According to the Invention

The aim of the invention is therefore to provide a composition having an improved effectiveness in combating irritable bowel syndrome, sleep, immunity and stress disorders.

For this purpose, the invention is aimed at a nutritional or therapeutic composition comprising:
at least one plant extract comprising at least 0.2% safranal by weight with respect to the total weight of the dry matter, measured by HPLC method, and
at least one probiotic bacterium and/or probiotic yeast.

The extract integrated in the composition can originate from (be obtained from) any plant containing safranal, in particular Crocus sativus, Centaurea sibthorpii, Centaurea consanguinea, Centaurea amanicola, Erodium cicutarium, Chinese green tea, Calycopteris floribunda, Crocus heuffelianus, Sambucus nigra, Gardenia jasminoides, Citrus limon, Cuminum cyminum L., Achillea distans. The plant containing the safranal from which the extract is obtained is preferably Crocus sativus.

The extract comprises at least 0.2% safranal by weight of dry matter, measured by HPLC method (High Performance Liquid Chromatography). The measuring method is extremely important, it being understood that, using another measuring method, in particular UV spectrometry (ISO 3632-2), the result obtained does not correspond to the actual safranal concentration, on account of the non-specificity of this method.

The method for analyzing molecules by HPLC is a method known to a person skilled in the art. It makes it possible to identity and quantify unitary molecules in a precise manner.

Preferably, the analysis method used for gauging the molecules contained in the extract according to the invention, in particular safranal, is a UHPLC method (Ultra High Performance Liquid Chromatography). This method makes it possible to further increase the resolution and the separation of the compounds, and to detect a plurality of compounds on the same chromatogram, from a single sample.

The extract can be obtained by any means that makes it possible to obtain at least 0.2% safranal by weight of dry matter of the extract. Preferably, the extract can be prepared according to the extraction method described in the patent FR 3054443.

According to a particularly suitable embodiment, the plant extract according to the invention is obtained by a method comprising the implementation of the following steps, carried out starting from the raw material saffron:

a. optionally drying,

b. crushing, preferably between 50 and 500 μm,

c. aqueous or hydroalcoholic extraction, or using an organic solvent,

d. impregnation of the obtained extract on a support,

e. encapsulation, preferably microencapsulation, and

f. heat treatment.

The step of heat treatment can be carried out at any time during this method, such as: after step b) and before step c), or between step c) and step d), preferably after step d), or after step e).

According to a particularly suitable embodiment, the step of heat treatment in the implementation of this method is a step of heat treatment in a sterilizer for at least 2 hours, even more preferably for at least 24 hours, at a temperature of between 30° C. and 95° C., even more preferably at a temperature of between 30° C. and 60° C.

The crushing can be performed using any suitable known means, in particular a granulating, pin or hammer mill, preferably a pin mill.

The extraction step may be carried out by any suitable known means.

In the case of aqueous extraction, the crushed material is introduced into water at the rate of 50 g/l.

In the case of hydroalcoholic extraction, the solvent can in particular be ethanol, preferably 60% v/v ethanol. The crushed material is introduced into the hydroalcoholic solution at the rate of 50 g/l.

In the case of extraction using an organic solvent, the solvent can in particular be methanol or ethyl acetate, preferably 30% v/v methanol. The crushed material is introduced into the organic solution at the rate of 100 g/l.

Following the extraction, the method can also comprise a step of acidification. This step consists in adding acid into the aqueous or hydroalcoholic solvent. It makes it possible to reduce the pH of the extraction solution to between 3 and 5. It can in particular be performed under the following conditions: addition of citric acid or hydrochloric acid into the hydroalcoholic solvent in order to adjust the pH to 4.

The step of impregnation on a support consists in adding a bulking agent into the extraction solution, i.e. in the liquid state. Said impregnation step in itself constitutes a first step of encapsulation of the extract, which may possibly be microencapsulation if the size of the particles is between 1 μm and 1000 μm, and more preferably between 30 μm and 500 μm, and even more preferably between 50 μm and 300 μm. It thus cannot simply be dry mixing with addition of an excipient or the support. The support or bulking agent can in particular be selected from the following constituents: maltodextrin, sugar, silica, gum arabic, preferably maltodextrin.

Following this impregnation step, the method may also comprise a step of emulsion and/or microencapsulation of the obtained extract. The microencapsulation can consist in coating during drying, or direct emulsion formation, inverse or double, optionally followed by drying. This microencapsulation step consists in high-speed stirring of the extraction solution containing the bulking agent and optionally the auxiliary agent. It can in particular be performed using auxiliary agents such as gum arabic, cyclodextrins or fats, vegetable waxes, hydrogenated or non-hydrogenated vegetable oils, proteins, peptides, dextrins, alginates, phospholipids. A double emulsion can be envisaged, using surfactants known to a person skilled in the art in order to allow solubilization in water of the extract initially encapsulated or microencapsulated, thus constituting double microencapsulation. According to a variant, the impregnation and microencapsulation steps are simultaneous. Preferably, the extract integrated in the composition comprises crocins and/or flavonoids derived from kaempferol, and/or derivatives of picrocrocin.

Crocetin and its derivatives, such as crocin, crocetin aglycons and/or glycolyzed crocetins can, as above, be extracted from Crocus sativus or Gardenia jasminoides, and it is largely responsible for the color of saffron.

Particularly preferably, the extract of Crocus sativus comprises terpenes such as safranal. The terpenes are volatile active compounds contained in saffron, and are known from the prior art for having an antimicrobial effect, and thus an effect against bacteria. For example, the article Nanasombat et al. 2014 describes the inhibition of two strains of Lactobacillus (plantarum and casei) and one strain of Saccharomyces cerevisiae using saffron pollen, in particular using safranal and crocins. The article Liu et al. 2017 describes an interaction of this kind between safranal and a strain of Escherichia Coli. The article Nadir et al. 2019 describes the effects of terpenes on the strains Staphylococcus aureus, Bacillus cereus, Entorococcus faecalis, Salmonella typhi, Pseudomonas aeruginosa, E. coli. More recently, the article by Ambrosio et al. 2020 describes the effect of terpenes on the strains E. coli and Lactobacillus rhamnosus.

However, surprisingly, the specific combination as well as the quantity of molecules contained in the present composition make it possible to achieve an effect on IBS, sleep, immunity and stress disorders in humans or animals, this effect being better than that of an extract of saffron alone, or of a probiotic bacterium or yeast alone. The effect on IBS, sleep, immunity and stress disorders in humans or animals can be achieved by increasing the concentration of bioactive metabolites (for example crocin aglycons, safranal) of the plant extract by means of the probiotic bacterium, or the improvement in the dysbiosis observed in some of these conditions.

Preferably, the quantity of plant extract by weight with respect to the total weight of the dry matter is between 0.5% and 20%, and the quantity of probiotic bacterium and/or probiotic yeast is between 80% and 99.5%.

Preferably, the extract may comprise, by weight of dry matter of the extract, measured by HPLC, at least 1% crocins and/or at least 500 ppm flavonoids derived from kaempferol, and/or at least 0.5% derivatives of picrocrocin.

Within the context of the invention, the extract according to any of the preceding embodiments is integrated in the composition and associated with at least one probiotic bacterium and/or probiotic yeast or a mixture. A mixture of this kind is made according to the techniques that are well known to a person skilled in the art.

Preferably, the probiotic bacterium and/or probiotic yeast may be dead, inactivated, semi-inactivated, or living. Said bacterium or yeast is preferably selected from the bacteria or yeast of the genus Lactobacillus, Bifidobacterium, Streptococcus, Saccharomyces, Enterococcus, Pediococcus and Escherichia. Even more preferably, the strain is selected from Lactobacillus, Bifidobacterium, and the mixture thereof.

The bacterium is preferably selected from the bacteria of the following species: Lactobacillus plantarum, Lactobacillus rhamnosus, Lactobacillus acidophilus, Lactobacillus delbrueckii ssp bulgaricus, Lactobacillus casei, Lactobacillus brevis, Lactobacillus fermentum, Lactobacillus reuteri, Lactobacillus salivarius, Lactobacillus paracasei, Lactobacillus helveticus, Bifidobacterium bifidum, Bifidobacterium infantis, Bifidobacterium coagulans, Bifidobacterium lactis, Bifidobacterium longum, Bifidobacterium animalis, Bifidobacterium breve, Bifidobacterium brevis, Bifidobacterium bifium, Lactococcus lactis, Streptococcus thermophilus, Streptococcus thermophiles, Streptococcus faecium, Enterococcus Faecalis, Enterococcus faecium Escherichia coli, Pediococcus acidilactici, Bacillus coagulans, Bacillus subtillis and the mixtures thereof.

The yeast is preferably selected from the yeasts of the following species: Saccharomyces cerevisiae, Saccharomyces boulardii and the mixtures thereof.

When the composition is intended to be used to improve digestion, the strain is selected from the bacteria and yeasts of the following species: Lactobacillus plantarum, Lactobacillus rhamnosus, Lactobacillus acidophilus, Lactobacillus delbrueckii ssp bulgaricus, Lactobacillus casei, Bifidobacterium bifidum, Bifidobacterium lactis, Bifidobacterium longum, Bifidobacterium animalis, Bifidobacterium bifium, Bifidobacterium breve, Pediococcus acidilactici, Streptococcus thermophilus, Saccharomyces cerevisiae, Streptococcus faecium, Enterococcus Faecalis, Enterococcus faecium, Escherichia coli and the mixtures thereof.

When the composition is intended to be used to improve sleep, the strain is selected from the bacteria of the following species: Lactobacillus fermentum, Lactobacillus rhamnosus, Lactobacillus plantarum, Bifidobacterium longum, Lactobacillus brevis, Lactobacillus casei, Lactobacillus helveticus and the mixtures thereof.

When Bifidobacterium longum is associated with Lactobacillus acidophilus, the composition is preferably intended to be used for improving digestion.

According to a particular embodiment, the composition according to the invention provides, daily, between 1×10⁵ and 9×10¹² CFU, preferably between 1×10⁷ and 9×10¹¹ of a probiotic bacterium and/or yeast and/or a mixture.

According to another embodiment, the composition may also comprise at least one vitamin selected from vitamins A, B1, B2, B3, B3/PP, B5, B6, B8/H, B9, B12, C, D, E, K and the mixture thereof.

The composition may also comprise at least one mineral selected from calcium, iodine, zinc, magnesium, copper, iron, selenium, potassium, fluorine, manganese, chloride, chromium, phosphorus and the mixture thereof, and/or at least one amino acid derivative selected from creatine, betaine and the mixture thereof.

The composition may also comprise at least one prebiotic and/or starch and/or at least one polyphenol in addition to those present in the plant extract or extracts. The prebiotics are preferably selected from FOS, GOS, and the polyphenols originate from red fruits, cocoa flavanols, olive oil or grapeseed oil.

According to another embodiment, the composition also comprises fats, such as phytosterols, omega 6 and omega 3, alpha-cyclodextrin, prunes, beta-glucan, lactulose, lactase, guar gum, melatonin, nuts, proteins, chitosan, red rice yeast, or carbon yeast.

When the composition is intended to be used to improve digestion, the composition may comprise fibers selected from wheat, oats, barley, rye, beetroot fibers, konjac, pectins, and chicory inulin.

Preferably, the quantity of fiber provided is between 3 g per 100 g of composition and 6 g per 100 g of composition, and/or it is between 1.5 g per 100 kcal of composition and 3 g per 100 kcal of composition.

The composition according to the invention or the plant extract is also impregnated on a support and/or microencapsulated in a support.

The support can in particular be selected from the following constituents: maltodextrin, sugar, silica, gum arabic, preferably maltodextrin.

The step of impregnation on a support of this kind consists in adding a bulking agent into the extraction solution. Thus, the impregnation is performed in solution, i.e. in the liquid state. This step is thus not carried out in a dry state by simple dry mixing with addition of an excipient or support, such as maltodextrin or dextrins.

The microencapsulation is particularly advantageous and makes it possible to stabilize and protect the saffron extract from its environment, in particular from the organism and bacteria present in the composition according to the invention. Thus, the terpenes such as the safranal and the saffron carotenoids such as the crocins and/or picrocrocins are protected, but also the bacteria.

Moreover, the microencapsulation makes it possible to trap the volatile and heat-sensitive active metabolites in the matrix, thus making it possible to obtain a saffron extract having high contents of active compounds. Finally, the microencapsulation makes it possible to improve the stability, the bioavailability, the organoleptic characteristics, and the use of said extract in food matrices, in particular with taste masking and a resistance to possible alterations to the compounds during the steps of producing a food product.

Thus, according to a first embodiment, the composition or the plant extract is microencapsulated in a food support selected from maltodextrin, gum arabic, hydrogenated or non-hydrogenated oil, a wax, alginates, starch of proteins or peptides. A microencapsulation method of this kind is well known to a person skilled in the art.

According to a second embodiment, the composition or the plant extract is impregnated on a support, preferably a food support. Such a method of impregnation on the support is described in the patent FR 3054443. The step of impregnation on a support consists in adding a bulking agent into the extraction solution. The support or bulking agent can in particular be selected from the following constituents: maltodextrin, sugar, silica, gum arabic, preferably maltodextrin.

According to a third embodiment, the composition or the plant extract is impregnated on a food support and then the composition or the plant extract is microencapsulated in a food support selected from maltodextrin, gum arabic, hydrogenated or non-hydrogenated oil, a wax, alginates, starch of proteins or peptides.

The composition may also be integrated into a food supplement present in the form of a capsule, powder, tablet, solution or chewing gum. The plant extract is preferably microencapsulated before being integrated into the food supplement. The invention thus also relates to a composition characterized in that it is a food supplement present in the form of a capsule, powder, tablet, solution or chewing gum.

According to another embodiment, the composition according to the invention may be integrated into a food product selected from dairy products, cereals, cereal products, and drinks. The invention thus also relates to a composition characterized in that it is a food product selected from dairy products, cereals, cereal products, and drinks.

### III. Use of a Composition According to the Invention/Composition for the Use Thereof

The composition according to the invention is intended for preventing and/or combating stress, mood disorders, sleep disorders, digestive disorders, immune disorders, impaired vision, erectile disorders, female libido disorders, joint disorders, cognitive impairments, cardiovascular disorders, disorders linked to premenstrual syndrome, disorders linked to the menopause, or for preventing and combating weight gain. Thus, the invention is aimed at the composition as described above, for these uses.

The composition according to the invention furthermore makes it possible to improve and reinforce the immune system, recovery, athletic performance, as well as oral health.

The composition can also be used for its anti-aging properties, in particular on the skin and in sun protection.

The mood disorders are preferably selected from depression, bipolar disorder, and dysthymia.

The disorders linked to stress are preferably selected from generalized anxiety disorder, panic disorder, social anxiety disorder, specific phobia, obsessive-compulsive disorder, post-traumatic stress disorder.

Preferably, the cognitive impairments are selected from Alzheimer's disease and/or Parkinson's disease and/or Huntington's disease and/or pathological cognitive decline and/or dementia and/or depression and/or schizophrenia and/or mental retardation and/or disorders associated with the post-menopausal condition in women, and/or cognitive disfunction syndrome (CDS).

The digestive disorders are preferably selected from irritable bowel syndrome, Crohn's disease, ulcerative colitis, celiac disease, Whipple's disease, dyspepsia, intestinal obstructions, and intestinal transit.

The impaired vision is preferably selected from age-related macular degeneration, glaucoma, diabetic retinitis.

The composition according to the invention is also intended for a therapeutic use for combating stress, mood disorders, sleep disorders, digestive disorders, immune disorders, impaired vision, erectile disorders, female libido disorders, joint disorders, cognitive impairments, cardiovascular disorders, disorders linked to premenstrual syndrome, disorders linked to the menopause, or for preventing and combating weight gain in humans or animals, for improving and reinforcing the immune system, recovery, athletic performance, as well as oral health, and for its anti-aging properties, in particular on the skin and in sun protection, in a healthy, i.e. not unwell, human or animal. The invention thus relates to these non-therapeutic uses of the composition as described above.

The invention is now illustrated by examples of a composition according to the invention, and results of tests demonstrating the absence of antimicrobial activity of safranal.

### IV. Example

### Example of a Composition Intended for Humans

Examples of a composition according to the invention are set out in table 1, below.

TABLE 1

| | | Example | | Example | | Example | | Example | |
|---|---|---|---|---|---|---|---|---|---|
| Ingredients | | 1 - Stress | | 2 - Sleep | | 3 - Digestion | | 4 - Digestion | |
| Saffron extract | | 30 | mg | 60 | mg | 30 | mg | 150 | mg |
| Probiotics | Lactobacillus acidophilus | X | | | | | | 5 × 10\hat{ }⁹ | CFU |
| | Lactobacillus delbrueckii | | | | | | | | |
| | Lactobacillus plantarum | X | | X | | X | | | |
| | Lactobacillus paracasei | X | | | | | | | |
| | Lactobacillus rhamnosus | X | | X | | | | | |
| | Lactobacillus bulgaricus | X | | | | | | | |
| | Lactobacillus gasseri | X | | | | | | | |
| | Lactobacillus fermentum | | | X | | | | | |
| | Bifidobacterium lactis | X | | | | X | | | |
| | Bifidobacterium longum | X | | | | X | | | |
| | Bifidobacterium bifidum | X | | | | | | | |
| | Bifidobacterium breve | X | | X | | X | | | |
| | Bifidobacterium infantis | X | | | | X | | | |
| | Saccharomyces boulardi | | | | | X | | | |
| | Mixture | 5.10\hat{ }10 | CFU | 4 × 10\hat{ }9 | CFU | 4.10\hat{ }9 | CFU | | |
| | Total probiotics | 159 | mg | 2,500 | mg | 175 | mg | 100 | mg |
| Vitamins | Vitamin A | | | | | | | | |
| | Vitamin B3 | | | | | 2.4 | mg | | |
| | Vitamin B12 | 0.375 | μg | 0.375 | μg | | | | |
| Minerals | Magnesium | 56.25 | mg | | | | | | |
| | Calcium | | | | | 120 | mg | | |
| Fibers | Barley | | | | | 3,000 | mg | | |
| Other | Melatonin | | | 1 | mg | | | | |
| | Activated carbon | | | | | 1,000 | mg | | |

| | | | | | | | | |
|---|---|---|---|---|---|---|---|---|
| TOTAL | 245.3 | mg | 2,561 | mg | 4,327 | mg | 2 | g |

| | | | | |
|---|---|---|---|---|
| Galenics | Capsule | Packet | Powder | Gummies |

The useful saffron extract in the composition according to the invention can be obtained according to various methods as set out below:

Extract According to the Invention 1

A first example of an extract is an extract obtained by carrying out a method consisting in implementing the following steps:
use of stigmata of Crocus sativus,
crushing using a pin mill, to a size of 250 μm,
hydroalcoholic extraction in 60% v/v ethanol, at a rate of 50 g of saffron per liter of hydroalcoholic solution,
impregnation on maltodextrin, introduced into the hydroalcoholic solution,
heat treatment in a sterilizer for 48 hours at 40° C.

The extract obtained is dosed in a plurality of molecules using the UHPLC method described in the preamble of the part relating to the examples.

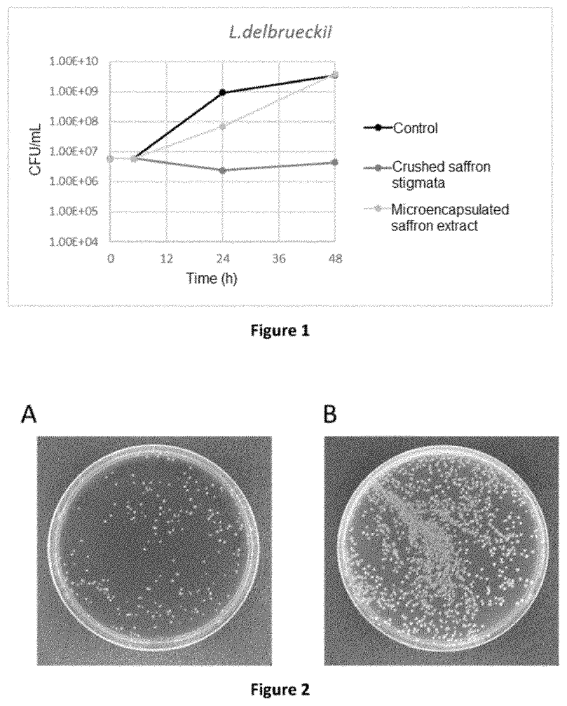

The chromatogram obtained is shown in FIG. 1.

The extract is characterized by:
a safranal concentration of 0.238%,
a crocin concentration of 3.96%,
a concentration of picrocrocin derivatives of 1.08%,
a concentration of flavonoids of 0.25%, and
particle size: 100%<1000 μm; 95%<500 μm.

Extract According to the Invention 2

A second example of an extract is an extract obtained by carrying out a method consisting in implementing the following steps:
use of stigmata of Crocus Sativus,
crushing using a pin mill, to a size of 250 μm,
acidified aqueous extraction using hydrochloric acid of pH 4,
impregnation on gum arabic introduced into the aqueous solution,
heat treatment in a sterilizer for 72 hours at 40° C.

The extract obtained is dosed in a plurality of molecules using the UHPLC method described in the preamble of the part relating to the examples.

The chromatogram obtained is shown in FIG. 2.

The extract is characterized by:
a safranal concentration of 0.73%,
a crocin concentration of 1.0%,
a concentration of picrocrocin derivatives of 2.93%,
a concentration of flavonoids of 0.57%, and
particle size: 100%<1000 μm; 95%<500 μm.

Extract According to the Invention 3

A third example of an extract is an extract obtained by carrying out a method consisting in implementing the following steps:
use of stigmata of Crocus Sativus,
crushing using a pin mill, to a size of 250 μm,
first heat treatment in a sterilizer for 2 hours to 6 hours at 105° C.,
second heat treatment in a sterilizer for 2 hours to 6 hours at 140° C.,
hydroalcoholic extraction in 60% v/v ethanol, at a rate of 50 g of saffron per liter of hydroalcoholic solution,
impregnation on maltodextrin, introduced into the hydroalcoholic solution,
drying, by lyophilization, of the liquid extract

The extract is characterized by:
a safranal concentration of 0.39%,
a crocin concentration of 2.31%,
a concentration of picrocrocin derivatives of 1.37%,
a concentration of flavonoids of 0.24%, and
particle size: 100%<1000 μm; 95%<500 μm.

Extract According to the Invention 4 (Multiple Microencapsulation)

A fourth example of an extract is a microencapsulated extract obtained by carrying out the method comprising the following steps:
use of the extract according to the invention 1,
solubilization of the extract in an aqueous phase by mechanical stirring,
primary inverse emulsification consisting in adding a mixture of vegetable oil and surfactant, and, simultaneously and at a high-performance, dispersion using a high-speed disperser system
secondary emulsification consisting in incorporating said primary emulsion in an aqueous mixture containing maltodextrin and gum arabic, and, at a high-performance, dispersion using a high-speed disperser system
vacuum drying of said double emulsion

The microencapsulated extract is characterized by:
a safranal concentration of 0.025% (after the double microencapsulation, and 0.238% after the first microencapsulation),
a crocin concentration of 0.557%,
a concentration of picrocrocin derivatives of 0.184%,
a concentration of flavonoids of 0.038%, and
particle size: 95%<500 μm.

Extract According to the Invention 5 (Multiple Microencapsulation)

A fifth example of an extract is a microencapsulated extract obtained by carrying out the method comprising the following steps:
use of the extract according to the invention 1,
solubilization of the extract in an aqueous phase by mechanical stirring,
primary inverse emulsification consisting in adding a mixture of vegetable oil and surfactant, and, simultaneously and at a high-performance, dispersion using a high-speed disperser system
secondary emulsification consisting in incorporating said primary emulsion in an aqueous mixture containing maltodextrin and gum arabic, and, at a high-performance, dispersion using a high-speed disperser system
recovery of said damp non-dried double emulsion.

The microencapsulated extract is characterized by:
a safranal concentration of 0.25%,
a crocin concentration of 3.56%,
a concentration of picrocrocin derivatives of 1.85%,
a concentration of flavonoids of 0.37%, and
particle size: 100%<1000 μm; 95%<500 μm; 90%<300 μm.

Extract According to the Invention 6

A sixth example of an extract is a microencapsulated extract obtained by carrying out the method comprising the following steps:
use of stigmata of Crocus sativus,
crushing using a granulating mill, to a size of 250 μm,
hydroalcoholic extraction in 60% v/v ethanol, at a rate of 500 g of saffron per liter of hydroalcoholic solution,
addition of water until 20% ethanol (v/v) is obtained,
impregnation on maltodextrin and gum arabic, introduced into the hydroalcoholic solution,
mechanical stirring,
primary inverse emulsification consisting in adding a mixture of sunflower oil and surfactant, and, simultaneously and at a high-performance, dispersion using a high-speed disperser system
secondary emulsification consisting in incorporating said primary emulsion in an aqueous mixture containing maltodextrin and gum arabic, and, at a high-performance, dispersion using a high-speed disperser system
vacuum drying of said double emulsion.

The microencapsulated extract is characterized by:
a safranal concentration of 0.31%,
a crocin concentration of 4.33%,
a concentration of picrocrocin derivatives of 1.95%,
a concentration of flavonoids of 0.38%, and
particle size: 100%<1000 μm; 95%<500 μm; 90%<300 μm.

Extract According to the Invention 7 (Multiple Microencapsulation)

A seventh example of an extract is a microencapsulated extract obtained by carrying out the method comprising the following steps:
use of stigmata of Crocus sativus,
crushing using a granulating mill, to a size of 250 μm,
hydroalcoholic extraction in 60% v/v ethanol, at a rate of 500 g of saffron per liter of hydroalcoholic solution,
addition of water until 20% ethanol (v/v) is obtained,
impregnation on maltodextrin and gum arabic, introduced into the hydroalcoholic solution,
mechanical stirring,
primary inverse emulsification consisting in adding a mixture of hemp oil and surfactant, and, simultaneously and at a high-performance, dispersion using a high-speed disperser system
secondary emulsification consisting in incorporating said primary emulsion in an aqueous mixture containing maltodextrin and gum arabic, and, at a high-performance, dispersion using a high-speed disperser system

The microencapsulated extract is characterized by:
a safranal concentration of 0.24%,
a crocin concentration of 4.15%,
a concentration of picrocrocin derivatives of 2.03%,
a concentration of flavonoids of 0.36%, and
particle size: 100%<1000 μm; 95%<300 μm.

The product obtained can be used as is for liquid applications, or dried in order to obtain a powder which can be used in dry applications.

The composition according to example 1 comprises 30 mg of an extract of saffron and 159 mg of a mixture of 11 probiotic bacterial strains in capsule form. The composition according to example 1 is obtained by mixing constituents in the conventional conditions known to a person skilled in the art.

The composition according to example 2 comprises 60 mg of an extract of saffron and 2500 mg of a mixture of 4 probiotic bacterial strains in the form of a powder contained in a sachet. The composition according to example 2 is obtained by mixing constituents in the conventional conditions known to a person skilled in the art.

The composition according to example 3 comprises 30 mg of an extract of saffron and 175 mg of a mixture of 6 probiotic bacterial strains in the form of a powder. The composition according to example 3 is obtained by mixing constituents in the conventional conditions known to a person skilled in the art.

The composition according to example 4 comprises 150 mg of an extract of saffron microencapsulated in a fat and 5×10^{\hat{ }9} CFU of Lactobacillus delbrueckii. The composition according to example 4 is obtained by mixing constituents in the conventional conditions known to a person skilled in the art.

The examples of the composition are obtained by mixing constituents in the conventional conditions known to a person skilled in the art.

### IV. Evaluation of the Effect of the Composition

Test 1:

The composition according to the invention exhibited a stability of the probiotic bacteria after incubation for 48 hours at 37° C., in contact with the plant extract comprising at least 0.2% safranal measured by HPLC. The composition according to the invention thus makes it possible to inhibit the natural antibacterial activity of the safranal.

Test 2: Evaluation of the Stability of a Probiotic with a Plant Extract Comprising at Least 0.2% Safranal.

The aim of this test is to demonstrate the compatibility of a probiotic bacterium with a plant extract comprising at least 0.2% safranal (measured by HPLC). The compatibility of a bacterium with a plant extract can be evaluated by determining the minimum inhibitory concentrations (MIC) of said plant extract vs. said bacterial strain. Thus, the higher the MICs, the more compatible the plant extract is with said bacterial strain. However, it is known from the prior art that the terpenes, such as safranal, contained in a plant extract of Crocus Sativus have an antimicrobial effect which, by definition, would make this type of plant extract incompatible with bacterial strains.

In order to evaluate the compatibility of probiotic bacteria with a plant extract comprising at least 0.2% safranal, the MICs in liquid medium were determined in vitro. In order to achieve this, the bacteria Lactobacillus delbrueckii and Bifidobacterium breve were isolated on MRS agar and incubated according to their optimal growth conditions for 48 hours. Then, for each strain, a 0.5 McFarland was carried out (the microbial cultures were diluted to obtain 105 to 10⁶ CFU/ml). In a sterile microplate and in a volume of 100 μl, increasing dilutions of 0.03 mg/L to 512 mg/L were carried out using an extract of saffron containing 0.2% safranal and microencapsulated, or crushed stigmata of Crocus Sativus. Then, a volume of 400 μL of microbial culture was added into each well. The plate was incubated for 48 hours in anaerobic condition before being read. Each test was performed 3 times. The results (Table 2) show that the MICs on the Lactobacillus delbrueckii and the Bifidobacterium breve are greater in the presence of a microencapsulated plant extract of Crocus Sativus compared with those in the presence of crushed stigmata of Crocus Sativus. Since the concentration of 512 mg/L was the highest concentration tested, these results show that a microencapsulated plant extract of Crocus Sativus is entirely compatible with Lactobacillus delbrueckii and Bifidobacterium breve. The plant extract of Crocus Sativus contained in the composition of the invention does not inhibit the growth of these probiotic bacteria, despite the presence of safranal.

TABLE 2
MIC in the presence of 2 different
plant extracts comprising safranal

| | | Saffron extract (0.2530% |
|---|---|---|
| | | safranal contained in |
| | Crushed saffron | example 4 of the |
| | stigmata | composition according to |
| | (0.2105% safranal) | the invention |
| Lactobacillus delbrueckii | 256 mg/L | >512 mg/L |
| Bifidobacterium breve | 256 mg/L | 512 mg/L |

Test 3: Evaluation of the Growth Kinetics of a Probiotic in the Presence of a Plant Extract Contained in the Composition of the Invention

The aim of this test is to confirm the results of the MICs on the Lactobacillus delbrueckii.

Proceeding from a colony on agar, of Lactobacillus delbrueckii, a liquid culture of bacteria was prepared and set to grow for 24 hours. A 50/50 mixture of microbial culture and culture medium was prepared in a Falcon tube. The fresh culture was recorded and used in exponential phase (0.5 McFarland). 3 ml of culture medium, 1 ml of microbial culture, and 1 ml of a microencapsulated saffron extract or crushed stigmata of Crocus Sativus, at 512 mg/L, were mixed in a Falcon tube. 100 μl of each preparation was plated on MRS agar and then incubated for 48 hours, at 37° C., anaerobically. The cultures and the petri dishes were incubated at 37° C., anaerobically, and recorded at TO, T5 h, T24 h and T48 h. The results (FIGS. 1 and 2) show that the bacterial growth is significantly inhibited after 48 hours in the presence of crushed stigmata of Crocus Sativus, while the bacterial growth is normal in the presence of a microencapsulated saffron extract.

Test 4: Measurement of the Consumption of Glucose by Yeasts in the Presence of a Plant Extract Comprising at Least 0.2% Safranal.

The aim of this test is to demonstrate that the consumption of glucose/fructose by the yeasts, reflecting their growth, is not altered in the presence of a plant extract comprising at least 0.2% safranal.

In order to achieve this, yeasts of the genus and species Saccharomyces cerevisiae were activated in water for 30 minutes at 37° C. at a ratio of 1 to 10 (5 g in 50 ml water). 500 mg of activated yeasts were added to 20 ml of a 25% glucose solution. Then, a microencapsulated saffron extract or crushed stigmata of Crocus Sativus at 625 mg/L were added to the yeasts. The yeasts, in the presence of plant extracts, were incubated at 30° C. The consumption of glucose/fructose was measured using an assay kit at TO and after 24 hours of incubation. A mixture of 730 μl of buffer reagent and 10 μl of each sample (diluted to 1/100) was made. Then, an absorbance reading (DO1) was performed at 340 nm. Then, 100 μl of the second reagent (100 μl) was added. A second absorbance reading (D02) was performed after 15 minutes of incubation. The glucose/fructose level is calculated in the following manner:

Csample (g/L)=C(standard)*(ΔDO sample/ΔDO standard)

Where DΔ0 sample=(DO2−DO1) sample−(DO2−D01) white, and ΔD0 standard=(DO2−DO1) standard−(DO2−DO1) white. The standard is a glucose/fructose solution at 5 g/l and which is used at 1.25 g/l

The consumption of glucose/fructose after 24 hours is then calculated, by expressing the glucose/fructose level after 24 hours with respect to T0.

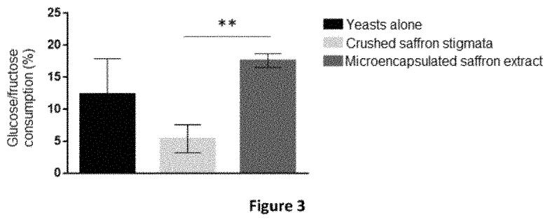

The results (FIG. 3) show that the yeasts in the presence of a microencapsulated saffron extract consume as much glucose/fructose as, and even slightly more than, the yeasts alone, 17.6% and 12.33% respectively, which shows that the yeasts exhibit normal growth in the presence of said extract. In contrast, in the presence of crushed stigmata of Crocus Sativus, the yeasts reduced their consumption of sugars (5.4%), which indicates that the growth of the yeasts had slowed. A significant difference between the consumption of sugars in the presence of a microencapsulated saffron extract and the consumption of sugars in the presence of non-microencapsulated stigmata of saffron (17.6% vs 5.4%). The composition according to the invention, i.e. an extract of saffron containing at least 0.2% microencapsulated safranal, allows for normal growth of yeasts, in contrast with a non-microencapsulated product containing at least 0.2% safranal.

1. A composition comprising:
at least one plant extract of Crocus sativus comprising at least 0.2% safranal by weight with respect to the total weight of the dry matter, measured by HPLC method, and
at least one probiotic bacterium and/or probiotic yeast,
characterized in that the extract is impregnated on a support and/or microencapsulated in a nutritional support selected from maltodextrin, gum arabic, a hydrogenated or non-hydrogenated oil, a wax, alginates, starch of proteins and peptides.

2. The composition according to claim 1, characterized in that the extract is microencapsulated in particles of sizes between 1 μm and 1000 μm.

3. The composition according to claim 1, characterized in that the extract of Crocus sativus comprises crocins and/or flavonoids derived from kaempferol, and/or derivatives of picrocrocin.

4. The composition according to claim 3, characterized in that the extract of Crocus sativus comprises terpenes.

5. The composition according to claim 4, characterized in that the extract comprises, by weight of dry matter of the extract, measured by HPLC, at least 1% crocins and/or at least 500 ppm flavonoids derived from kaempferol, and/or at least 0.5% derivatives of picrocrocin.

6. The composition according to claim 1, characterized in that the probiotic bacterium and/or probiotic yeast is dead, inactivated, semi-inactivated, or living.

7. The composition according to claim 1, characterized in that the probiotic bacterium and/or probiotic yeast is selected from the bacteria and/or yeast of the genus Lactobacillus, Bifidobacterium, Bacillus, Streptococcus, Saccharomyces, Enterococcus, Pediococcus and Escherichia.

8. The composition according to claim 7, characterized in that the extract of Crocus sativus is obtained from stigmata and/or petals and/or bulbs of Crocus sativus.

9. The composition according to claim 1, characterized in that it also comprises at least one vitamin selected from vitamins A, B1, B2, B3, B3/PP, B5, B6, B8/H, B9, B12, C, D, E and K.

10. The composition according to claim 1, characterized in that it also comprises at least one mineral selected from calcium, iodine, zinc, magnesium, copper, iron, selenium, potassium, fluorine, manganese, chloride, chromium and phosphorus, and/or at least one amino acid derivative selected from creatine and betaine.

11. The composition according to claim 1, characterized in that it also comprises at least one prebiotic and/or starch and/or at least one polyphenol in addition to those present in the plant extract or extracts.

12. The composition according to claim 11, characterized in that the prebiotics are selected from FOS, GOS and the polyphenols in addition to those present in the plant extract or extracts originate from red fruits.

13. The composition according to claim 1, characterized in that the composition is a food supplement present in the form of a capsule, powder, tablet, solution or chewing gum.

14. The composition according to claim 1, characterized in that the composition is a food product present in the form of dairy products, cereals, cereal products and drinks.

15. The composition according to claim 1, for use thereof in the prevention and/or treatment of digestive disorders, sleep disorders, immune disorders, impaired vision, mood disorders, erectile disorders, female libido disorders, joint disorders, cognitive impairments, cardiovascular disorders, premenstrual syndrome, disorders related to the menopause, stress-related disorders, to aid weight loss, to provide an anti-aging action in particular on the skin, and sun protection, in humans and animals.

16. The composition according to claim 15, useful in the prevention and/or treatment of digestive disorders selected from irritable bowel syndrome, Crohn's disease, ulcerative colitis, celiac disease, Whipple's disease, dyspepsia, intestinal obstructions, and intestinal transit.

17. The composition of claim 16, characterized in that it also comprises fibers selected from wheat, oats, barley or rye.
